(19) United States
(12) Patent Application Publication
(10) Pub. No.: US 2018/0116861 A1
(43) Pub. Date: May 3, 2018

(54) UNDERGARMENT PAD

(71) Applicants: Kerstin Gilberg, St. Augustine, FL (US); Julie Wright, St. Augustine, FL (US); Nancy Nelson, St. Augustine, FL (US)

(72) Inventors: Kerstin Gilberg, St. Augustine, FL (US); Julie Wright, St. Augustine, FL (US); Nancy Nelson, St. Augustine, FL (US)

(21) Appl. No.: 15/335,589

(22) Filed: Oct. 27, 2016

(51) Int. Cl. A61F 5/453; A61F 5/44; A61F 13/537

(52) U.S. Cl. CPC A61F 5/453; A61F 13/53708; A61F 5/4401

(57) ABSTRACT

A male genital guard that includes: an outer surface; an inner surface; and layers of absorption between the inner surface and outer surface. Preferably, the inner surface is adapted for placement around a penis and the guard is used with an adult diaper.

### BACKGROUND OF THE INVENTION

### Field of Invention

The present invention relates to padding for the male penis for adult diaper users.

### Description of Related Art

More individuals are utilizing adult diapers due to incontinence or medical conditions. Most of these individuals must wear the adult diaper to avoid accidents due to inability to control urine flow. Use of diapers requires constant changing and removal of wet diapers either by the caregiver or the adult user. Some diapers are very effective in removing the moisture from the contact area near the skin and genitals of the users. However, there's a period of time where the user may experience some discomfort due to the accumulation of urine within the diaper. It is imperative that the genitals and skin be clear of any urine to avoid urinary tract infections that normally occur due to the use of adult diapers. This issue is especially a concern with users that are bedridden who require caregivers to remove and change diapers on a regular basis. Male diaper users may experience additional aggravation due to the limited amount of products that separate the penis from the rest of the inner portion of the diaper. As a result, it would be advantageous to have a padding designed to cover the penis at all times to increase the effectiveness of the diaper and reduce chances of infection.

### SUMMARY OF THE INVENTION

The present invention relates to a male genital guard that includes: an outer surface; an inner surface; and layers of absorption between the inner surface and outer surface. Preferably, the inner surface is adapted for placement around a penis and the guard is used with an adult diaper.

### BRIEF DESCRIPTION OF DRAWINGS

FIG. 1 depicts the penis guard in a closed position.

FIG. 2 depicts an open position for the penis guard in accordance with the present invention.

### DETAILED DESCRIPTION

The present invention relates to a padding and guard that is used in conjunction with an adult diaper to guard the user's penis while using an adult diaper. The guard according to present invention opens and wraps around the length of the penis providing a guard of the surface area of the penis. The male genitals are normally exposed when using an adult diaper and therefore are subject to contamination and occurrence of urinary tract infections.

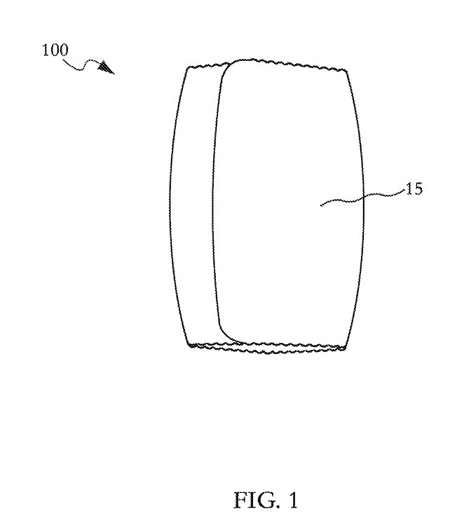

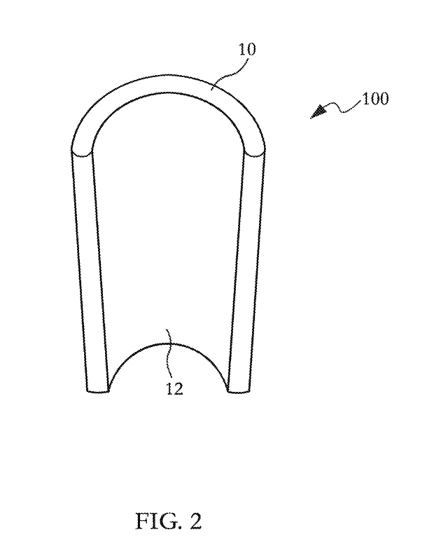

In reference to FIG. 1, a male genital guard (100) in accordance with the present invention is depicted. The male genital guard (100) is in a closed position and outer surface (15) is shown and depicted. The guard (100) includes multiple layers of absorption to keep moisture away from the male genitals wrapped within the guard (100). The guard (100) is used as an addition to an adult diaper to absorb and wick away moisture from the male genitals. In reference to FIG. 2, the genital guard (100) is shown in an open position. The inner surface (12) is depicted and layered absorption (10) is also shown in FIG. 2. This layered absorption (10) absorbs moisture and therefore keeps moisture on the outer perimeter of the guard (100) while the inner surface (12) remains dry in an effort to protect the male genital from wetness while using an adult diaper.

Other alternative uses of the present invention may include used as a bandage in order to address wound care. Other embodiments may include padding for a nursing mother, disposable urine collection sampling, first aid kits and implementation into emergency rooms, doctor offices or other medical facilities. The guard padding may also be used simply as a wipe to absorb any moisture in any environment such as a kitchen or bathroom. The instant invention has been shown and described in what it considers to be the most practical and preferred embodiments. It is recognized, however, that departures may be made there from within the scope of the invention and that obvious modifications will occur to a person skilled in the art.

What is claimed is:

1. A male genital guard comprising:
a. an outer surface;
b. an inner surface; and
c. layers of absorption between the inner surface and outer surface.

2. The male genital guard according to claim 1, where the inner surface is adapted for placement around a penis.

3. The male genital guard according to claim 1, where the guard is used with an adult diaper.
